(19) United States
(12) Patent Application Publication
(10) Pub. No.: US 2026/0256355 A1
(43) Pub. Date: Sep. 3, 2026

(54) SYSTEM AND METHOD FOR DETECTING GLAUCOMA

(71) Applicant: MEDIOS TECHNOLOGIES PTE LTD, Singapore (SG)

(72) Inventors: Divya RAO PARTHASARATHY, Ellicott City, MD (US); Florian Michael SAVOY, Singapore (SG); Chao-Kai HSU, Singapore (SG); Bhargav SOSALE, Singapore (SG)

(21) Appl. No.: 18/842,769

(22) PCT Filed: Oct. 26, 2023
(86) PCT No.: PCT/IB2023/060814

§ 371 Date: Aug. 29, 2024

(30) Foreign Application Priority Data
Oct. 26, 2022 (IN) 202211061028

(51) Int. Cl. A61B 3/14; A61B 3/00; G06T 7/00

(52) U.S. Cl. CPC A61B 3/14; A61B 3/0025; G06T 7/0012; G06T 2207/20081; G06T 2207/30041

(57) ABSTRACT

Approaches for glaucoma detection are described. In an example, a region of interest (ROI) portion of an input eye image, wherein the input eye image corresponds to a subject eye under evaluation for detecting presence of glaucoma, is obtained. A detection model pipeline is thereafter used, wherein the detection model pipeline is trained based on training data comprising one of training characteristic information corresponding to plurality of input eye image characteristics, and images associated with glaucoma, and wherein the detection model pipeline. The detection model pipeline is used to extract a characteristic information from the ROI portion of the input eye image to determine vertical cup-to-disc ratio (vCDR). Thereafter, classification output denoting probability of presence of glaucoma in the subject eye is obtained. Based on the above parameters, presence of glaucoma within the subject eye is determined.

### BACKGROUND

The eyes are human body's most highly developed sensory organ which acquire a greater portion of working brain as compared to the other sensory organs. By protecting or via regular screening of the eyes, the odds such as blindness and vision loss may be reduced which may be caused by any developing eye disease such as glaucoma. Glaucoma is a chronic disease which affects the optic nerves of the eye. If glaucoma is left untreated, it may lead to permanent damage of the optic nerves and causes blindness. Glaucoma can be diagnosed by performing a number of tests that include but not limited to gonioscopy, tonometry, visual field test, OCT and pachymetry. However, none of the above disclosed tests have been found to be individually sufficient to provide accurate results for large scale screening of population at minimal cost.

### BRIEF DESCRIPTION OF FIGURES

Systems and/or methods, is accordance with examples of the present subject matter are now described and with reference to the accompanying figures, in which:

FIG. 1A-1B illustrates a training system for training a glaucoma detection model, as per an example;

FIG. 2 illustrates a glaucoma detection system for determining presence of glaucoma in an input eye image, as per another example;

FIG. 3 illustrates a method for training a glaucoma detection model, as per an example; and

FIGS. 4A-4B illustrates a method for determining presence of glaucoma in an input eye image, based on a trained glaucoma detection model, as per an example.

### DETAILED DESCRIPTION

Eyes are the most used sensory organ among the five senses of the human body and eyes perceives most of the information about the world. Eye includes a retina at its back, which on illumination with light, cause the photoreceptors to turn the light into electrical signals. These electrical signals travel from the retina through the optic nerve to the brain for further processing. Such electric signals are then processed by the brain to create a visual feed or perception of surrounding objects which we see as images or videos.

An individual may, in certain instances, suffer from different vision disorders. Examples of vision disorder may include, but are not limited to, blurred vision (refractive errors), age-related macular degeneration, glaucoma, cataract, diabetic retinopathy, etc. One such visual disorder is glaucoma, a leading cause of blindness. Glaucoma is a progressive optic neuropathy with characteristic structural changes in the optic nerve head. The damage caused by glaucoma cannot be reversed, but proper and timely detection of glaucoma may help slow or prevent vision loss.

Various conventional approaches provide clinical information to diagnose glaucoma. One of such diagnostic tests is funduscopic examination of the optic disc and retinal nerve fibre layer in which an ophthalmologist analyses the structural changes of optic disc and retinal nerve fibre to ascertain presence of glaucoma. It may be noted that, glaucomatous changes are manifested by tissue loss at the neuro-retinal rim and enlargement of the optic nerve excavation, a non-physiological discrepancy between the optic nerve excavations in the two eyes, haemorrhages at the edge of the optic disc, thinning of the retinal nerve fiber layer, and parapapillary tissue atrophy. Other diagnostic techniques include morphometric techniques which enable quantitative examination of the optic disc and measurement of the retinal nerve fiber layer and neuro-retinal rim with optical coherence tomography (OCT).

The above-described techniques or other such diagnostic methods require a specialist ophthalmologist and expensive equipment. As may be understood, presence of such highly specialized medical practitioner and equipment is limited to tertiary level health care centre which may be far away from the reach of rural population, which may be the case particularly for developing nations, such as India. To perform screening of large population with minimal cost, there is a need for a system which performs automatic detection of glaucoma having an on-the-edge operable configuration to reduce cost and time of operation of such system.

Approaches for detecting presence of glaucoma based on an input eye's retinal image, are described. The input eye image may be an image of the eye of a patient which is under screening. Such input eye image may be either stored in a database repository or may be captured by a camera device. In one example, input eye image, corresponding to a subject eye, which is to be screened for detecting glaucoma, is obtained. Once obtained, the input eye image may be processed to obtain a region of interest (ROI) portion of the input eye image. In one example, the ROI portion may be obtained by using a localization model. In another example, the ROI portion may be obtained after assessing a quality of the input eye image. It may be noted that assessing quality prior to obtaining the ROI portion is not essential. In an example, the ROI portion may be obtained from an input eye image and then subject to a quality assessment process. In either example, the quality of the image may be assessed using a quality model.

Once the ROI portion of input eye image is obtained, the same may be processed based on a segmentation model to obtain characteristic information. Such characteristic information corresponds to plurality of eye image characteristics. Examples of such eye image characteristics include, but are not limited to, cup-to-disc ratio (CDR). Further other examples may include size, color, and integrity of the neuro retinal rim (NRR), size and shape of the optic cup, shape and configuration of the vessels in the optic disc, indicator indicating presence of the laminar dot sign in the cup, optic disc hemorrhages, structural changes in peripaillary region, RNFL defects, and many more. All such examples would still be withing the scope of the present subject matter. In one example, the characteristic information may be used as a measurement parameter for ascertaining presence of glaucoma, as described subsequently. In another example, the characteristic information relied on may be a cup-to-disc ratio (CDR) or a vertical CDR (vCDR).

Continuing with the present example, the ROI portion may be further processed based on a classification model to provide a probability value indicative of presence of glaucoma. It may be understood, a high value of the probability value would correspond to a high likelihood of glaucoma whereas a low probability value would correspond to a less likelihood of glaucoma. In addition to the probability value, a visualization output may also be generated. In one example, the visualization output may be in the form of an activation map. The activation map thus obtained may indicate one or more salient regions of the ROI portion. The salient regions, as may be noted, may correspond to regions that may be afflicted by optic nerve damage.

Proceeding further, the ROI portion may be further processed based on a thickness detection model to detect a Retina Nerve Fiber Layer (RNFL) thickness, as exhibited by the subject eye. To this end, the ROI portion may be initially processed to obtain a set of sub-images. In one example, the sub-images may be in the form of quadrants (i.e., four equal parts) or other sectors. Each of the sub-images may be further processed based on the thickness detection model to obtain candidate RNFL thickness values corresponding to each sub-image (e.g., each quadrant). The candidate RNFL thickness values may be averaged to obtain an averaged RNFL thickness value.

As explained above, the ROI portion may be processed separately to obtain the characteristic information (e.g., the vCDR), the probability value indicative of presence of glaucoma and the averaged RNFL thickness value. Once the aforementioned parameters are determined, the same may be processed to provide an assessment as to whether glaucoma is present in the subject eye. Based on the processing of characteristic information (e.g., the vCDR) and the probability value indicative of presence of glaucoma, the input eye image is categorized under one of possible health categories. In an example, the input eye image may be categorized into a healthy eye, a glaucomatous/disc suspected eye (which may be referred as non-urgent category), and an urgent glaucomatous eye. As per the determination, appropriate medical treatment may then be prescribed. In an example, in addition to the above parameters, averaged RNFL thickness value may also be utilized for categorizing the input eye image under any one of the possible health categories. These and other aspects have been discussed in further detail later in the present description.

It may be noted that the above-mentioned determinations involving obtaining the characteristic information (e.g., the vCDR), the probability value indicative of presence of glaucoma or the averaged RNFL thickness value may involve a variety of models such as the segmentation model, the classification model, and the thickness detection model. In one example, each of the aforementioned models are machine learning based models. In an example, the machine learning model may be a deep learning model. Although having been described as unique or separate models, the segmentation model, the classification model, and the thickness detection model may be implemented as a detection model pipeline for the detection of glaucoma in the subject eye. It may also be noted that the detection model pipeline may include other types of models (e.g., a localization model, quality model, and others) for performing one or more intermediate functions, without deviating from the scope of the present subject matter.

The machine learning models within the detection model pipeline may be trained on a variety of training information. For example, the segmentation model may be trained based on training images with segmented portions defining the optic discs and the optic cups. In a similar manner, the classification model may be trained on images (i.e., the ROI portions) associated with glaucoma and images not associated with glaucoma, or through attributes that may be obtained through clinical history, comprehensive eye examination and investigational modalities that include but not limited to optical coherence tomography, visual fields, intraocular pressure measurements, pachymetry etc.. The thickness detection model in turn may be trained based on the images with confirmed or verified RNFL thickness values. Such values may have been confirmed using a variety of techniques, such as Optical coherence tomography (OCT). Although the training has been described in the context of the segmentation model, the classification model and the thickness detection model, such similar training procedures may be performed for other models that may be implemented within the detection model pipeline. Such processes would still fall within the scope of the present subject matter without limitation.

The present approaches overcome the above-mentioned technical advantages. For example, the above-mentioned approaches may be implemented in a single device for effective glaucoma screening. Since no specialized equipment or skill is required, a system implementing the present approaches is mobile, cost-effective, and accurate for the purposes of glaucoma detection. For example, an implementing system allows for screening without expert knowledge and is performable on portable retinal camera itself, while ensuring a desired and functional level of accuracy.

The explanation provided above and the examples that are discussed further in the current description are exemplary only. For instance, some of the examples may have been described in which only one image is considered, either in training or in inference stage. However, the current approaches may be adopted for other instances or situations as well, such as a set of input eye images, a set of training eye images may be used, or such without deviating from the scope of the present subject matter.

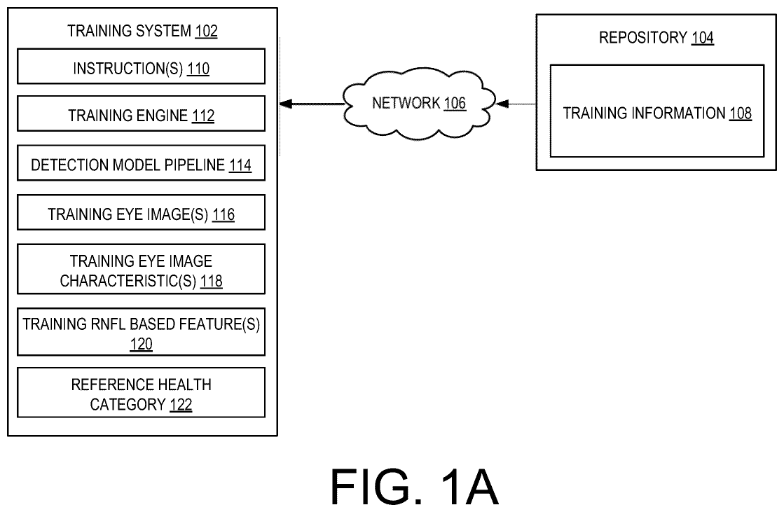

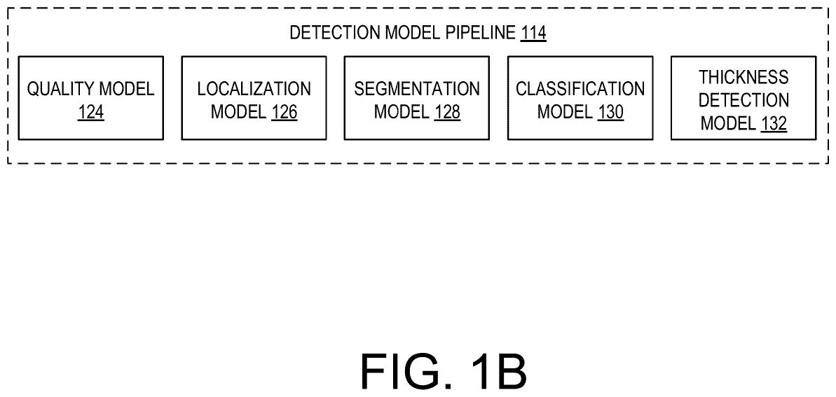

The manner in which models implemented within the detection model pipeline are trained and used for identifying presence of glaucoma in the input eye image is explained in detail with respect to FIGS. 1A-4B. While aspects of described systems may be implemented in any number of different electronic devices, environments, and/or implementation, the examples are described in the context of the following example device(s). In another example, the aspects of the present subject matter may also be implemented by a standalone device having executable instructions. It may be noted that drawings of the present subject matter shown here are for illustrative purposes and are not to be construed as limiting the scope of the subject matter claimed.

FIG. 1A illustrates a training system 102 comprising a processor or memory (not shown), for training models within the detection model pipeline. In an example, the training system 102 (referred to as system 102) may be communicatively coupled to a repository 104 through a network 106. The repository 104 may further include training information 108. The training information 108 may include training data that may be used for training the detection model pipeline.

In another example, along with plurality of images, the training information 108 may further include training eye image characteristics and corresponding health category of each of the plurality of images. In an example, these pluralities of images are those images which are captured previously while manual screening of the patient with corresponding health category annotated. In an example, the training eye image characteristics may include size, color, and integrity of the neuro-retinal rim (NRR), coordinates of the disc center specified in the training images (for disc localization purposes), size and shape of the optic cup, cup-to-disc ratio (CDR), shape and configuration of the vessels in the optic disc, indicator indicating presence of the laminar dot sign in the cup, optic disc hemorrhages, structural changes in peripapillary region, RNFL defects or loss, and various combinations thereof. Although depicted as being obtained from a single repository, such as repository 104, the training information 108 may also be obtained from multiple other sources without deviating from the scope of the present subject matter. In such cases, each of such multiple repositories may be interconnected through a network, such as network 106.

The network 106 may be a private network or a public network and may be implemented as a wired network, a wireless network, or a combination of a wired and wireless network. The network 106 may also include a collection of individual networks, interconnected with each other and functioning as a single large network, such as the Internet. Examples of such individual networks include, but are not limited to, Global System for Mobile Communication (GSM) network, Universal Mobile Telecommunications System (UMTS) network, Personal Communications Service (PCS) network, Time Division Multiple Access (TDMA) network, Code Division Multiple Access (CDMA) network, Next Generation Network (NGN), Public Switched Telephone Network (PSTN), Long Term Evolution (LTE), and Integrated Services Digital Network (ISDN).

The system 102 may further include instructions 110 and a training engine 112. In an example, the instructions 110 are fetched from a memory and executed by a processor included within the system 102. The training engine 112 may be implemented as a combination of hardware and programming, for example, programmable instructions to implement a variety of functionalities. In examples described herein, such combinations of hardware and programming may be implemented in several different ways. For example, the programming for the training engine 112 may be executable instructions, such as instructions 110. Such instructions may be stored on a non-transitory machine-readable storage medium which may be coupled either directly with the system 102 or indirectly (for example, through networked means). In an example, the training engine 112 may include a processing resource, for example, either a single processor or a combination of multiple processors, to execute such instructions. In the present examples, the non-transitory machine-readable storage medium may store instructions, such as instructions 110, that when executed by the processing resource, implement training engine 112. In other examples, the training engine 112 may be implemented as electronic circuitry.

The instructions 110, when executed by the processing resource, cause the training engine 112 to train the detection model pipeline 114 based on the training information 108. The system 102 may further include a training eye image(s) 116, a training eye image characteristic(s) 118, a training RNFL based feature(s) 120, and a reference health category 122. In an example, the system 102 may obtain training information 108 corresponding to a single training eye image from the repository 104, and the information pertaining to that is stored as training eye image(s) 116, training eye image characteristic(s) 118, training RNFL based feature(s) 120 and reference health category 122 in the system 102.

As described previously, the detection model pipeline 114 may further include a plurality of machine learning models. An example of such machine learning models include deep learning models. For the sake of explanation, the current approaches for detection of glaucoma has been described with the different steps being performed using one or more deep learning models, as examples. Although the present examples have been described in relation to deep learning models, the aforementioned approaches may also be implemented using other machine-learning models. It may also be noted that any explanation provided in conjunction with deep learning models is applicable to other machine learning models, without limitations and without deviating from the scope of the present subject matter. Such examples have not been described for sake of brevity. The manner in which the training of the plurality of the models within the detection model pipeline 114 may be performed is further described in conjunction with FIG. 1B. FIG. 1B depicts example deep learning models that may be implemented within the detection model pipeline 114. In one example, the detection model pipeline 114 may include a quality model 124, a localization model 126, a segmentation model 128, a classification model 130 and a thickness detection model 132. It may be noted that the detection model pipeline 114 may include other deep learning models (not shown in FIG. 1B) as well for implementing various other functions. It may also be the case that one or more models may be implemented so as to perform a combination of one or more functions. Such variations and combinations would still be examples of the present subject matter without limitations.

With respect to training the quality model 124, the training eye image(s) 116 may be used wherein the training eye image(s) 116 may include images having higher resolution, contrast, clarity, or other such attributes. The localization model 126 in turn may be trained on training eye image(s) 116 which identify the portions of image corresponding to the optic disc and corresponding coordinates defining the position of the optic disc within the training eye image(s) 116. Still further, the segmentation model 128 may be trained based on images with segmented portions defining the optic discs and the optic cups through training eye image characteristic(s) 118. In an example, the eye image characteristics such as training eye image characteristic(s) 118 corresponding to the training eye image(s) 116 may include size, color, and integrity of the neuro-retinal rim (NRR), size and shape of the optic cup, cup-to-disc ratio (CDR), shape and configuration of the vessels in the optic disc, indicator indicating presence of the laminar dot sign in the cup, structural changes in peripaillary region, optic disc hemorrhages, RNFL loss, and many more.

In a similar manner, the classification model 130 may be trained on images (i.e., the ROI portions) associated with glaucoma and images not associated with glaucoma as part of the training image(s) 116 associated with a corresponding reference health category 122. The thickness detection model 132 in turn may be trained based on training RNFL based feature(s) 120 which correspond to images with confirmed or verified RNFL thickness values. As discussed previously, such values may have been confirmed using techniques, such as Optical coherence tomography (OCT). Although the RNFL based feature(s) 120 are explained in the context of RNFL thickness, other RFNL related features or attributes may also be utilized without deviating from the scope of the present subject matter. Such other features include but are not limited to appearance, size and shape of the RNFL. For example, loss of RNFL may be manifested by way of loss of appearance of RNFL striations which are nothing but the retinal ganglion axons that may be packed together in bundles, but viewable as normal dark-light striations during a fundus examination. Change in size may also be indicated of RNFL defects. For instance, variation in size of the RNFL may occur due to slit defects, wedge defects, or in some cases, complete loss as well. It may be noted that these example features are only indicative and should not be considered as limiting the scope of the present subject matter in any way.

As will be discussed subsequently, the quality model 124, the localization model 126, the segmentation model 128, the classification model 130, and the thickness detection model 132 when trained may be used to determine a variety of parameters based on which presence of glaucoma within a subject eye may be ascertained. In an example, once trained, the detection model pipeline 114 may be utilized for categorizing an input eye image as one of a plurality of health categories. Examples of such health categories include, but are not limited to, healthy eye, glaucoma suspect eye, and urgent glaucoma eye.

The training of the quality model 124, the localization model 126, the segmentation model 128, the classification model 130, and the thickness detection model 132 may be performed in any order and may be performed and different instants. As may be understood, although one or more common training datasets may be used, the training of any one of the deep learning models in the detection model pipeline 114 is independent from the training of another model.

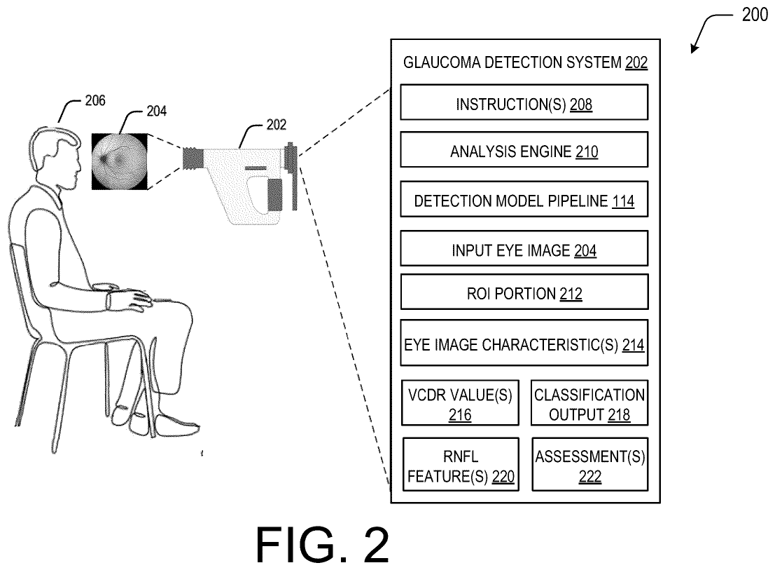

Once trained, the detection model pipeline 114 may be used to categorize the input eye image under one of the possible health categories. The manner in which the detection model pipeline 114 may be used for detection of glaucoma within the subject eye is further described in conjunction with FIG. 2.

FIG. 2 illustrates an environment 200 with a glaucoma detection system 202 for determining a health category of an input eye image 204 of a patient. In an example, the glaucoma detection system 202 (referred to as system 202) includes a mobile phone, tablet, or any other portable computing device. In an example, the portable computer device attached onto the system 202 is capable of capturing fundus images of the patient. The input eye image 204 may be an image of an eye of the patient who is under screening for the diagnosis of glaucoma. In an example, the input eye image 204 is a fundus image. In an example, the system 202 may analyze a plurality of eye image characteristics of the input eye image 204 based on the trained detection model pipeline 114

Similar to the system 102, the system 202 may further include instructions 208 and an analysis engine 210. In an example, the instructions 208 are fetched from a memory and executed by a processor included within the system 202. The analysis engine 210 may be implemented as a combination of hardware and programming, for example, programmable instructions to implement a variety of functionalities. In examples described herein, such combinations of hardware and programming may be implemented in several different ways. For example, the programming for the analysis engine 210 may be executable instructions, such as instructions 208. Such instructions 208 may be stored on a non-transitory machine-readable storage medium which may be coupled either directly with the system 202 or indirectly (for example, through networked means). In an example, the analysis engine 210 may include a processing resource, for example, either a single processor or a combination of multiple processors, to execute such instructions. In the present examples, the non-transitory machine-readable storage medium may store instructions, such as instructions 208, that when executed by the processing resource, implement analysis engine 210. In other examples, the analysis engine 210 may be implemented as electronic circuitry.

In one example, the analysis engine 210 may utilize the trained detection model pipeline 114 to ascertain whether glaucoma is present within the subject eye, to which the input eye image 204 may correspond to. It may be noted that the detection model pipeline 114 may be trained by way of the approach discussed in conjunction with FIGS. 1A-1B. As also described previously, the detection model pipeline 114 may further include trained quality model 124, the localization model 126, the segmentation model 128, the classification model 130, and the thickness detection model 132.

The system 202 may further include an ROI portion 212, eye image characteristic(s) 214, vCDR 216, classification output 218, RNFL feature(s) 220 and assessment(s) 222. It may be noted that the aforesaid data elements are generated by the analysis engine 210 using the detection model pipeline 114 and in response to the execution of the instruction(s) 208. These aspects and further details are discussed in the following paragraphs.

In operation, an input eye image 204 of an eye of a subject patient who is under screening for the detection of glaucoma, may be captured. The input eye image 204 may be captured through any image sensing sub-system that may be present within the system 202. In an example, the image sensing sub-system may be a retinal camera device which is either installed on the system 202 itself or may be removably integrated with the system 202.

Once the input eye image 204 is obtained, the analysis engine 210 may assess quality of the input eye image 204 using the trained detection model pipeline 114. In one example, analysis engine 210 may utilize the trained quality model 124 of the detection model pipeline 114 for ascertaining quality of the input eye image 204. If the image quality of the input eye image 204 is acceptable, the input eye image 204 may be processed by the analysis engine 210 using the detection model pipeline 114. In an example, if the input eye image 204 is not of acceptable quality, the user 206 may be prompted to capture the input eye image 204, again. In such instances, the user 206 may initiate the capture of another input eye image 204 or may choose to proceed with the initially captured input eye image 204. Bot such examples are complimentary and as such have no impact on the scope of the present subject matter. It may be understood that ascertaining the quality of the input eye image 204 may rely on various features or attributes of the input eye image 204, as detected by the quality model 124. It may be noted that the user 206 may elect to proceed with subsequent process based on the input eye image 204 without assessing its quality, without deviating from the scope of the present subject matter.

The input eye image 204 (once determined as acceptable as the case may be), may be further processed by the analysis engine 210 using the trained detection model pipeline 114 to identify portion of the input eye image 204 which includes the optic disc. In one example, the analysis engine 210 may utilize the trained localization model 126 of the detection model pipeline 114 to detect the portion of the input eye image 204 corresponding to the optic disc. To this end, the analysis engine 210 may, using the localization model 126, determine positional coordinates of a portion of the image which corresponds to the optic disc. Based on the positional coordinates this determined using the trained localization model 126, the analysis engine 210 may accordingly crop the input eye image 204 to obtain the ROI portion 212. It may be noted that the ROI portion 212 is such that the optic disc is centered therein.

It may be noted that the trained localization model 126 may identify the position of the optic disc in the input eye image 204 through image analysis techniques performed on the input eye image 204, as per an example. For example, regions of the input eye image 204 with higher degree of illumination will denote the presence of optic disc location effectively. It may be noted that the aforesaid example is one of the many other approaches that may be adopted by the localization model 126 of the detection model pipeline 114. Any other approach may also be used by the localization model 126 without deviating from the scope of the present subject matter.

The ROI portion 212 may be further processed by the analysis engine 210 using the trained detection model pipeline 114. In one example, analysis engine 210 using the trained segmentation model 128 may process the ROI portion 212 to obtain one or more eye image characteristic(s) 214. Examples of the eye image characteristic(s) 214 include, but is not limited to, cup-to-disc ratio (CDR), size, color, and integrity of the neuroretinal rim (NRR), size and shape of the optic cup, shape and configuration of the vessels in the optic disc, indicator indicating presence of the laminar dot sign in the cup, optic disc hemorrhages, structural changes in peripaillary region, and tRNFL loss or defect. In one example, the analysis engine 210 may determine the vCDR 216 based on the determined eye image characteristic(s) 214. For example, the cup-to-disc ratio may be utilized to measure and compute the vertical cup-to-disc ratio or the vCDR 216. The vCDR 216 thus obtained may be stored in the system 202. In one example, the analysis engine 210, may determine dimensions of the optic cup and optic disc by segmenting outline of the optic disc and the optic cup from the ROI portion of the input eye image 204.

In addition, the processing of the ROI portion 212 by the analysis engine 210 using the trained segmentation model 128, the ROI portion 212 may also be analyzed based on the trained classification model 130. In one example, the ROI portion 212 may be processed by the analysis engine 210 using the classification model 130. The analysis performed based on the classification model 130 is to ascertain a probability or likelihood that the subject eye under consideration has glaucoma or not. The outcome of the analysis performed by the analysis engine 210 using the classification model 130 of the detection model pipeline 114 may be stored as classification output 218. The analysis performed by the deep learning classification model 130 may involve image analysis comprising extracting and processing one or more features of the ROI portion 212.

In one example, the classification output 218 may denote a probability of presence of glaucoma in the subject eye under consideration. As described previously, the probability determined by using the classification model 130 is based on analysis of the ROI portion 212 and depicts whether the subject eye has glaucoma or not. In one example, the classification output 218 may further include a visual output in the form of an activation map. As may be understood, the activation map thus generated may depict or highlight salient regions, for example where optic disc damage is present, or where RNFL defects may be present, within the ROI portion 212. It may also be the case that that the activation map may indicate other types of defects, without deviating from the scope of the present subject matter.

Continuing further, in an example, the ROI portion 212 may be further processed by the analysis engine 210 using the trained thickness detection model 132 to determine one or more RNFL feature(s) 220. To this end, the analysis engine 210 may initially divide the ROI portion 212 into a number of sub-images. For example, the analysis engine 210 may divide the ROI portion 212 into four equal quadrants. It may be noted that the number of sub-images may change depending on level of accuracy that is intended for the glaucoma detection. To this end, the analysis engine 210 may cause to divide the ROI portion 212 into equally sized segments.

In one example, the sub-images may be so formed, such that each of the quadrants correspond to nasal, temporal, inferior, and superior fields of vision. The respective sub-images may then be processed to determine one or more RNFL features corresponding to each quadrant. Thereafter, the analysis engine 210 may average the RNFL features determined for each sub-image to obtain the averaged RNFL feature which is stored as RNFL feature(s) 220. The RNFL feature(s) 220 thus determined may be stored for further analysis as will be discussed in the coming paragraphs. An example of the RNFL features includes RNFL thickness.

With the vCDR 216 and the classification output 218 thus obtained, presence of glaucoma within the subject eye may be determined. In one example, the analysis engine 210 based on the vCDR 216 and the classification output 218 may generate an assessment, such as the assessment(s) 222, indicating the presence or absence of glaucoma within the subject eye. In one example, the assessment(s) 222 thus generated may be based on one or more predefined rules and specified conditions based on which the different parameters, namely, the vCDR 216 and classification output 218, are to be processed to provide the assessment(s) 222. Although explained in the context of the present example, the analysis engine 210 may generate the assessment(s) 222 through other techniques as well, without deviating from the scope of the present subject matter.

It may be noted that the assessment(s) 222 may be generated by further considering the RNFL feature(s) 220 along with the vCDR 216 and the classification output 218. In an example, with the vCDR 216, the classification output 218 and the RNFL feature(s) 220 obtained, presence of glaucoma within the subject eye may be determined. In one example, the analysis engine 210 based on the vCDR 216, the classification output 218 and the RNFL feature(s) 220 may generate an assessment, such as the assessment(s) 222, indicating the presence or absence of glaucoma within the subject eye. In one example, the assessment(s) 222 thus generated may be based on one or more predefined rules and specified conditions based on which the different parameters, namely, the vCDR 216, classification output 218 and the RNFL feature(s) 220, are to be processed to provide the assessment(s) 222.

It may be noted that the assessment(s) 222 thus generated may be used to provide a further referral for treatment, or other intervention, as may be required. For example, the assessment(s) 222 may be indicative of a diagnosis of glaucoma. The assessment(s) 222 may indicate one of the following states: normal, disc suspect or glaucoma. Based on the state represented by the assessment(s) 222, appropriate action may be taken. Although explained as being obtained by considering vCDR 216, classification output 218 (and the RNFL feature(s) 220, such as, RNFL thickness in certain instances), the assessment(s) 222 may be obtained by considering any one or more of the above parameters without deviating from the scope of the present subject matter. Such examples would still fall within the scope of the present subject matter, without any limitation.

Once all the results of processing based on the ROI portions are obtained, the analysis engine 210 takes these results either alone or in any possible combination to determine the presence of glaucoma in the input eye image 204 or categorize the input eye image 204 as one of the health categories. The identified resultant category for the input eye image 204 then may be displayed on the display device of the system 202 to indicate the health category of the patient under screening so that further steps of treatment are practiced for curing the disease. In an example, the assessment(s) 222 being displayed on a per eye, per patient basis, or as a combination thereof. For example, the segmentation maps and activation maps are shown per eye, but the glaucoma categorization is shown per patient by taking the worst eye image.

In another example, the system 202 may be communicatively coupled to a central computing server through a network (not shown in FIG. 2). The network may be a private network or a public network and may be implemented as a wired network, a wireless network, or a combination of a wired and wireless network, and may be similar to the network 106 (as depicted in FIG. 1A). All the above disclosed steps which may be performed by the analysis engine 210 of the system 202, may be implemented or performed by the central computing server on behalf of the system 202 to reduce computing load on edge of the network.

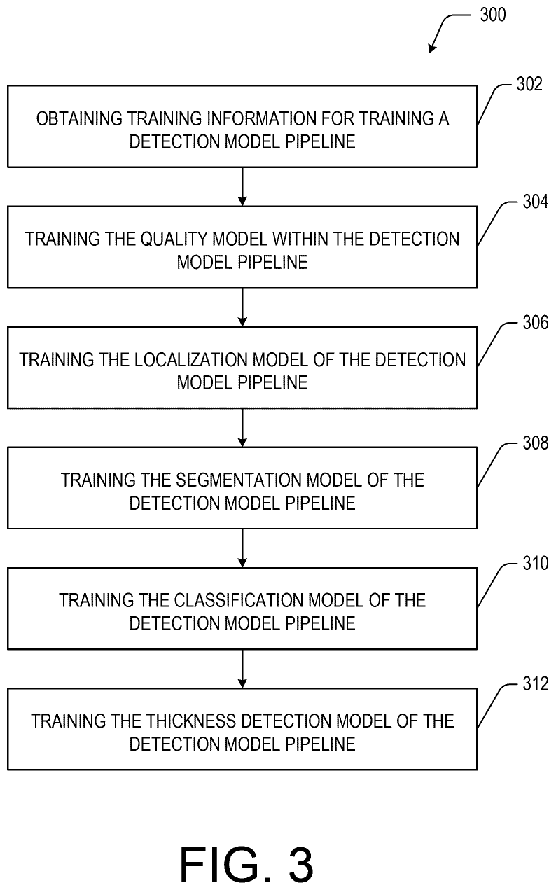

FIG. 3 illustrates an example method 300 for training a glaucoma detection model, in accordance with examples of the present subject matter. The order in which the above-mentioned method is described is not intended to be construed as a limitation, and some of the described method blocks may be combined in a different order to implement the method, or alternative method.

Furthermore, the above-mentioned method may be implemented in a suitable hardware, computer-readable instructions, or combination thereof. The steps of such method may be performed by either a system under the instruction of machine executable instructions stored on a non-transitory computer readable medium or by dedicated hardware circuits, microcontrollers, or logic circuits. For example, the method may be performed by a training system, such as system 102. In an implementation, the method may be performed under an “as a service” delivery model, where the system 102, operated by a provider, receives programmable code. Herein, some examples are also intended to cover non-transitory computer readable medium, for example, digital data storage media, which are computer readable and encode computer-executable instructions, where said instructions perform some or all the steps of the above-mentioned method.

In an example, the method 300 may be implemented by the system 102 for training one or more glaucoma detection models based on a training information, such as training information 108. At block 302, training information for training a detection model pipeline may be obtained. For example, a training system 102 may obtain training information 108. The training information 108 may be obtained through a repository, such as the repository 104. In one example, the training information 108 may include a training eye image(s) 116, a training eye image characteristic(s) 118, and a training RNFL based feature(s) 120 based on which different models in the detection model pipeline 114 are to be trained. In another example, the detection model pipeline 114 may include quality model 124, localization model 126, segmentation model 128, classification model 130 and thickness detection model 132.

At block 304, the quality model within the detection model pipeline may be trained. In one example, the training engine 112 may train the quality model 124 using the training eye image(s) 116, wherein the training eye image(s) 116 may include images having higher resolution, contrast, clarity, or other such attributes.

At block 306, the localization model of the detection model pipeline may be trained. For example, the training engine 112 of the training system 102 may train the localization model 126 based on training eye image(s) 116. In one example, the training eye image(s) 116 identifies the portions of image corresponding to the optic disc and corresponding coordinates defining the position of the optic disc within the training eye image(s) 116.

At block 308, the segmentation model of the detection model pipeline may be trained. For example, the training engine 112 of the training system may train the segmentation model 128 based on images with segmented portions defining the optic discs and the optic cups. In an example, the eye image characteristics such as training eye image characteristic(s) 118 corresponding to the training eye image(s) 116 may include size, color, and integrity of the neuro-retinal rim (NRR), size and shape of the optic cup, cup-to-disc ratio (CDR), shape and configuration of the vessels in the optic disc, indicator indicating presence of the laminar dot sign in the cup, structural changes in peripaillary region, optic disc hemorrhages, RNFL loss, and many more.

At block 310, the classification model of the detection model pipeline may be trained. For example, the training engine 112 of the training system may train the classification model 130 based on images (i.e., the ROI portions) associated with glaucoma and images not associated with glaucoma as part of the training image(s) 116 associated with a corresponding reference health category 122.

At block 312, the thickness detection model of the detection model pipeline may be trained. For example, the training engine 112 of the training system may train the thickness detection model 132 based on training RNFL based feature(s) 120 which correspond to images with confirmed or verified RNFL thickness values. As discussed previously, such values may have been confirmed using techniques, such as Optical coherence tomography (OCT). Although the RNFL based feature(s) 120 are explained in the context of RNFL thickness, other RFNL related features or attributes may also be utilized without deviating from the scope of the present subject matter. Examples of such other features include but are not limited to size, color, and shape of the RNFL.

As will be discussed subsequently, the quality model 124, the localization model 126, the segmentation model 128, the classification model 130, and the thickness detection model 132 when trained may be used to determine a variety of parameters based on which presence of glaucoma within a subject eye may be ascertained. In an example, once trained, the detection model pipeline 114 may be utilized for categorizing an input eye image as one of a plurality of health categories. Examples of such health categories include, but are not limited to, healthy eye, glaucoma suspect eye, and urgent glaucoma eye.

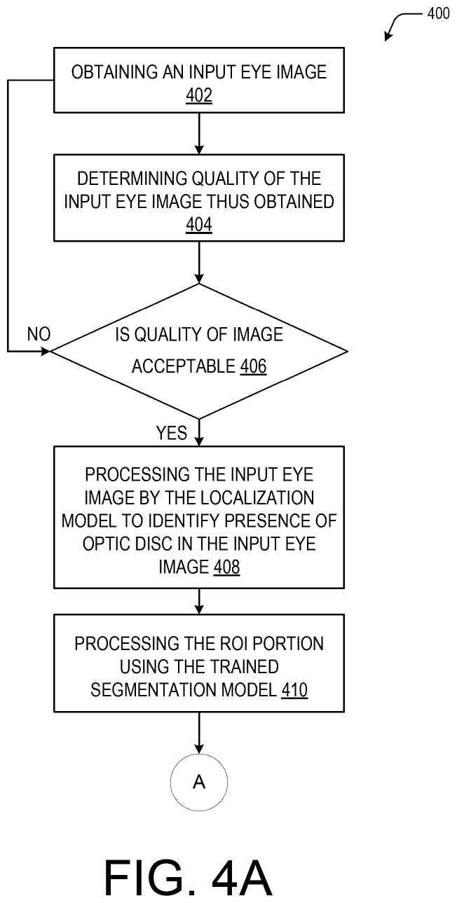

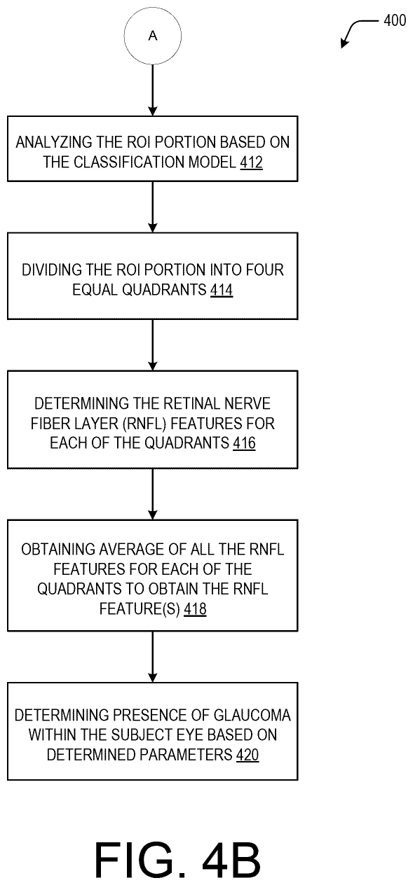

FIGS. 4A-4B illustrates example method 400 for categorizing an input image under one of health categories. Similar to FIG. 3, the order in which the above-mentioned method is described is not intended to be construed as a limitation, and some of the described method blocks may be combined in a different order to implement the method, or alternative method. Based on the present approaches as described in the context of the example method 400, the eye image characteristics of an input eye image is analyzed based on the trained detection model pipeline 114.

Further, the above-mentioned method 400 may be implemented in a suitable hardware, computer-readable instructions, or combination thereof. The steps of such method may be performed by either a system under the instruction of machine executable instructions stored on a non-transitory computer readable medium or by dedicated hardware circuits, microcontrollers, or logic circuits. For example, the method may be performed by a glaucoma detection system, such as system 202. In an implementation, the method may be performed under an “as a service” delivery model, where the system 202, operated by a provider, receives programmable code. Herein, some examples are also intended to cover non-transitory computer readable medium, for example, digital data storage media, which are computer readable and encode computer-executable instructions, where said instructions perform some or all the steps of the above-mentioned method.

At block 402, an input eye image is obtained. For example, the system 202 may obtain an image of an eye of a suspected patient who is under screening for the detection of glaucoma. The image of the eye, i.e. input eye image 204, is stored as input eye image 204 in the system 202. In an example, the input eye image 204 is captured by the system 202 using a retinal camera device which is either installed on the system 202 itself or connected externally to the system 202. In an example, there are other external hardware equipment needs to be installed along with the system 202 to capture or get the retinal view of an eye of a person. In another example, the input eye image 204 may be obtained from a database repository (not shown in FIG. 2) storing samples of eye images to be tested for detecting presence of glaucoma.

At block 404, the quality of the input eye image thus obtained may be determined. For example, once the input eye image 204 is obtained, the analysis engine 210 may assess quality of the input eye image 204 using the trained detection model pipeline 114. In one example, analysis engine 210 may utilize the quality model 124 of the detection model pipeline 114 for ascertaining quality of the input eye image 204.

At block 406, a determination may be made to ascertain whether the image quality of the input eye image is acceptable or not. For example, if the image quality of the input eye image 204 is acceptable (‘Yes’ path from block 406), the input eye image 204 may be processed by the analysis engine 210 using the detection model pipeline 114, as will be described in later steps. If, however, the input eye image 204 is not of acceptable quality (‘No’ path from block 406), the user 206 may be prompted for capturing the input eye image 204 again (prior to block 402). It may be understood that ascertaining the quality of the input eye image 204 may rely on various features or attributes of the input eye image 204, as detected by the quality model 124. It may be noted that the steps recited in blocks 404 and 406 are optional—in some cases the user 206 may elect to proceed with the input eye image 204 without assessing the quality of the same. Such examples would still fall within the purview of the present subject matter.

At block 408, the input eye image may be further processed by the localization model to identify the presence of optic disc in the input eye image. For example, the input eye image 204 once determined as acceptable, may be further processed by the analysis engine 210 using the trained detection model pipeline 114 to identify portion of the input eye image 204 which includes the optic disc. In one example, the analysis engine 210 may utilize the trained localization model 126 of the detection model pipeline 114 to detect the portion of the input eye image 204 corresponding to the optic disc. To this end, the analysis engine 210 may, using the localization model 126, determine positional coordinates of a portion of the image which corresponds to the optic disc. Based on the positional coordinates this determined using the trained localization model 126, the analysis engine 210 may accordingly crop the input eye image 204 to obtain the ROI portion 212. It may be noted that the ROI portion 212 is such that the optic disc is centered therein. In one example, the trained localization model 126 may identify the position of the optic disc in the input eye image 204 through image analysis techniques performed on the input eye image 204, as per an example. It may be noted that the aforesaid example is one of the many other approaches that may be adopted by the localization model 126 of the detection model pipeline 114. Any other approach may also be used by the localization model 126 without deviating from the scope of the present subject matter.

In an example, the ROI portion 212 may be analyzed by the analysis engine 210 to ascertain the quality of the ROI portion 212 using the quality model 124. In an example, the quality assessment may entail determining whether the ROI portion 212 conforms with one or more attributes, for example, brightness, clarity, contrast, etc. In another example, the analysis engine 210 may process the ROI portion 212 using the quality model 124 to determine whether the optic disc is present within the ROI portion 212. Based on the determination, the subsequent processes may proceed or the user 204 may be prompted to capture another input eye image 204, without deviating from the scope of the present subject matter.

At block 410, the ROI portion may be further processed using the trained segmentation model. For example, analysis engine 210 using the trained segmentation model 128 may process the ROI portion 212 to obtain one or more eye image characteristic(s) 214. Examples of the eye image characteristic(s) 214 include, but is not limited to, cup-to-disc ratio (CDR), size, color, and integrity of the neuroretinal rim (NRR), size and shape of the optic cup, shape and configuration of the vessels in the optic disc, indicator indicating presence of the laminar dot sign in the cup, optic disc hemorrhages, structural changes in peripaillary region, and tRNFL loss or defect. In one example, the analysis engine 210 may determine the vCDR 216 based on the determined eye image characteristic(s) 214. For example, the cup-to-disc ratio may be utilized to measure and compute the vertical cup-to-disc ratio or the vCDR 216. The vCDR 216 thus obtained may be stored in the system 202. In one example, the analysis engine 210, may determine dimensions of the optic cup and optic disc by segmenting outline of the optic disc and the optic cup from the ROI portion of the input eye image 204.

At block 412, the ROI portion may also be analyzed based on the classification model. For example, the ROI portion 212 may be processed by the analysis engine 210 using the classification model 130. The analysis performed based on the classification model 130 is to ascertain a probability or likelihood that the subject eye under consideration has glaucoma or not. In one example, the outcome of the analysis performed by the analysis engine 210 using the classification model 130 of the detection model pipeline 114 may be stored as classification output 218. In one example, the classification output 218 may denote a probability of presence of glaucoma in the subject eye under consideration. As described previously, the probability determined by using the classification model 130 is based on analysis of the ROI portion 212 and depicts whether the subject eye has glaucoma or not. In one example, the classification output 218 may further include a visual output in the form of an activation map. As may be understood, the activation map thus generated may depict or highlight salient regions where optic damage is present, within the ROI portion 212.

At block 414, the ROI portion may be divided into equal sub-images. For example, the ROI portion 212 may be further processed by the analysis engine 210 may divide the ROI portion 212 into a plurality of sub-images. In such cases, the sub-images may In an example, the analysis engine 210 may process the ROI portion 212 to split the same into four equal quadrants. The quadrants may be so formed, such that each of the quadrants correspond to nasal, temporal, inferior, and superior fields of vision.

At block 416, the retinal nerve fiber layer (RNFL) features for each of the quadrants may be determined. For example, the analysis engine 210 may process each of the quadrants to determine one or more RNFL features, corresponding to each quadrant. An example of the RNFL features includes RNFL thickness.

At block 418, the average of all the RNFL features for each of the quadrants may be determined. For example, the analysis engine 210 may average the RNFL features determined for each quadrant to obtain the averaged RNFL feature, which is stored as RNFL feature(s) 220. The RNFL feature(s) 220 thus determined may be stored for further analysis.

At block 420, presence of glaucoma within the subject eye may be determined based on the determined parameters. For example, the analysis engine 210 may determine presence of glaucoma based on the vCDR 216 and the classification output 218 thus obtained. In one example, the analysis engine 210 based on the vCDR 216 and the classification output 218 may generate an assessment, such as the assessment(s) 222, indicating the presence or absence of glaucoma within the subject eye. In one example, the assessment(s) 222 thus generated may be based on one or more predefined rules and specified conditions based on which the different parameters, namely, the vCDR 216 and classification output 218, are to be processed to provide the assessment(s) 222.

It may be noted that the analysis engine 210 may generate the assessment(s) 222 by further considering the RNFL feature(s) 220 along with the vCDR 216 and the classification output 218. In an example, with the vCDR 216, the classification output 218 and the RNFL feature(s) 220 obtained, presence of glaucoma within the subject eye may be determined. In one example, the analysis engine 210 based on the vCDR 216, the classification output 218 and the RNFL feature(s) 220 may generate an assessment, such as the assessment(s) 222, indicating the presence or absence of glaucoma within the subject eye. In one example, the assessment(s) 222 thus generated may be based on one or more predefined rules and specified conditions based on which the different parameters, namely, the vCDR 216, classification output 218 and the RNFL feature(s) 220, are to be processed to provide the assessment(s) 222

It may be noted that the assessment(s) 222 thus generated may be used to provide a further referral for treatment, or other intervention, as may be required. For example, the assessment(s) 222 may be indicative of a diagnosis of glaucoma. The assessment(s) 222 may indicate one of the following states: normal, disc suspect or glaucoma. Based on the state represented by the assessment(s) 222, appropriate action may be taken. Although explained as being obtained by considering vCDR 216, classification output 218 and the RNFL feature(s) 220 (e.g., RNFL thickness), the assessment(s) 222 may be obtained by considering any one or more of the above parameters without deviating from the scope of the present subject matter. Such examples would still fall within the scope of the present subject matter, without any limitation.

Once all the results of processing based on the ROI portions are obtained, the analysis engine 210 takes these results either alone or in any possible combination to determine the presence of glaucoma in the input eye image 204 or categorize the input eye image 204 as one of the health categories. The identified resultant category for the input eye image 204 then may be displayed on the display device of the system 202 to indicate the health category of the patient under screening so that further steps of treatment are practiced for curing the disease.

Although examples for the present disclosure have been described in language specific to structural features and/or methods, it is to be understood that the appended claims are not necessarily limited to the specific features or methods described. Rather, the specific features and methods are disclosed and explained as examples of the present disclosure.

1. A system comprising:
a processor; and
an analysis engine coupled to the processor, wherein the analysis engine is for:
obtaining a region of interest (ROI) portion of an input eye image, wherein the input eye image corresponds to a subject eye under evaluation for detecting presence of glaucoma;
using a detection model pipeline, wherein the detection model pipeline is trained based on training data comprising one of training characteristic information corresponding to plurality of input eye image characteristics, and images associated with glaucoma, and wherein the detection model pipeline is for:
extracting a characteristic information from the ROI portion of the input eye image to determine vertical cup-to-disc ratio (vCDR) corresponding to the subject eye in the input eye image;
obtaining classification output denoting probability of presence of glaucoma in the subject eye; and
determining presence of glaucoma within the subject eye based on the vCDR and classification output.

2. The system as claim in claim 1, wherein the analysis engine is for using the detection model pipeline to determine retinal nerve fiber layer (RNFL) features based on the input eye image, wherein the detection model pipeline is trained also based on retinal nerve fiber layer (RNFL) based features.

3. The system as claimed in claim 1, wherein the plurality of eye image characteristics comprises size, color, and integrity of the neuroretinal rim (NRR), size and shape of the optic cup, cup-to-disc ratio (CDR), shape and configuration of the vessels in the optic disc, indicator indicating presence of the laminar dot sign in the cup, optic disc hemorrhages, structural changes in peripaillary region, and RNFL defects.

4. The system as claimed in claim 1, wherein the analysis engine is for:
assessing a quality of the input eye image;
rejecting the input eye image upon determining a quality score to be less than a threshold quality score; and
prompting a user to obtain or capture new input eye image.

5. The system as claimed in claim 1, wherein the analysis engine is for further:
ascertaining presence of an optic disc in the input eye image upon determining a quality score to be greater than a threshold quality score, wherein presence of the optic disc is ascertained by a neural network based machine learning;
determining a set of coordinates of the center of the optic disc in the input eye image; and
performing cropping of the input eye image based on the set of coordinates of the center of the optic disc to obtain the ROI portion of the input eye image.

6. The system as claimed in claim 1, wherein the detection model pipeline comprises a plurality of deep learning models selected from a group consisting of a quality model, a localization model, a segmentation model, classification model, and a RFNL thickness detection model.

7. The system as claimed in claim 1, wherein to categorize the input eye image as one of the health categories, the analysis engine using the trained detection model pipeline is for:
cropping the ROI portion of the input eye image to obtain a set of four quadrants portions based on the determined set of coordinates of the center of the optic disc in the input eye image;
processing the set of four quadrants portions to determine a Retinal Nerve Fiber Layer (RNFL) thickness value for each of the quadrant portions;
determining an average thickness of RNFL across the quadrant portions based on the individual RNFL thickness of the quadrants; and
categorize categorizing the input eye image as one of the healthy eye, the glaucoma suspect eye, and the urgent glaucoma eye based on the average thickness of the RNFL.

8. The system as claimed in claim 1, wherein the classification output comprises an activation map depicting salient regions within the ROI portion where optic damage is present.

9. A method comprising:
obtaining a training information comprising a training eye image and information comprising training characteristic information corresponding to plurality of input eye image characteristics, images associated with glaucoma and retinal nerve fiber layer (RNFL) features corresponding to a training dataset; and
training a detection model pipeline based on training data comprising one of training characteristic information corresponding to plurality of input eye image characteristics, images associated with glaucoma and retinal nerve fiber layer (RNFL) features.

10. The method as claimed in claim 9, wherein training characteristic information comprises size, color, and integrity of the neuroretinal rim (NRR), size and shape of the optic cup, cup-to-disc ratio (CDR), shape and configuration of the vessels in the optic disc, indicator indicating presence of the laminar dot sign in the cup, optic disc hemorrhages, structural changes in peripaillary region, and RNFL defects.

11. The method as claimed in claim 10, wherein the detection model pipeline when trained based on the characteristic information is for determining a vertical cup-to-disc ratio (CDR).

12. The method as claimed in claim 9, wherein the detection model pipeline comprises a plurality of deep learning models selected from a group consisting of a quality model, a localization model, a segmentation model, classification model, and a thickness detection model.

13. The method as claimed in claim 9, wherein each training RNFL visual feature comprises a training feature value, wherein the training RNFL visual features comprises size, color, and shape of the RNFL.

14. The method as claimed in claim 9, wherein the detection model pipeline when trained is for assessing quality of one of the input eye image and region of interest (ROI) portion of an input eye image, wherein the input eye image corresponds to a subject eye under evaluation for detecting presence of glaucoma.

15. The method as claimed in claim 9, wherein the detection model pipeline when trained is to categorize the subject eye as one of healthy eye, glaucoma suspect eye, and urgent glaucoma eye.
